(19) United States
(12) Patent Application Publication
(10) Pub. No.: US 2004/0133109 A1
(43) Pub. Date: Jul. 8, 2004

(54) Systems and methods for acoustic thermal imaging

(75) Inventors: Robert J. Crowley, Sudbury, MA (US); Isaac Ostrovsky, Wellesley, MA (US)

Correspondence Address: ORRICK, HERRINGTON & SUTCLIFFE, LLP, 4 PARK PLAZA, SUITE 1600, IRVINE, CA, 92614-2558, US

(73) Assignee: Scimed Life Systems, Inc.

(21) Appl. No.: 10/339,164

(22) Filed: Jan. 7, 2003

(51) Int. Cl. A61B 8/00

(52) U.S. Cl. 600/438; 600/459

(57) ABSTRACT

A thermal acoustic system is a medical imaging device, such as a catheter, adapted to measure the temperature of portions of a lumen of a living being. In one embodiment, the catheter includes a transducer configured to receive a thermally generated acoustic wave when located within the lumen and output a first signal corresponding to the intensity of the received thermally generated acoustic wave. In one embodiment, the thermal acoustic system also displays a temperature measurement, or relative temperatures, of the lumen mapped onto an image of the topology of the lumen. The catheter can further include an elongated, tubular member configured to allow the transducer to move within the elongated member and a control system configured to move the transducer such that it can radially and axially scan the temperature of the lumen.

### FIELD OF THE INVENTION

The field of the invention relates generally to thermal imaging, and more particularly to the acoustic thermal imaging of a lumen of a living being.

### BACKGROUND INFORMATION

Catheters are tools commonly employed to help diagnose and treat medical conditions by allowing access to remote and otherwise unreachable locations within a body. A wide range of medical procedures can be performed with a catheter, such as imaging, angioplasty and the release of therapeutic agents into the body. Catheters provide particular advantages when used to gain access to a body lumen such as a blood vessel or artery.

Atherosclerosis is one of the most prevalent degenerative coronary and vascular diseases in the world. It is characterized by a constriction and hardening of blood vessels due to increased lipid deposition on the vessel wall, resulting in platelet activation, calcification and fibrosis. These lipid deposits are further characterized in two forms—occlusive plaque and vulnerable plaque. Atherosclerosis caused by occlusive plaque is typically treated with the aid of a catheter.

In diagnosing and/or treating atherosclerosis, it is desirable to use an imaging catheter to obtain images of the vessel wall. For instance, an image of the inner surface of a blood vessel can be created using an imaging catheter based on ultrasound or light imaging techniques. The ultrasound catheter contains an ultrasound transducer and is connected to a control and imaging system. The control system sends an excitation signal to the transducer which causes it to vibrate. This vibration generates acoustic waves that emanate out from the transducer until they contact the inner surface of the lumen. The acoustic waves echo off the inner surface and back to the transducer. When the waves reach the transducer, they cause the transducer to vibrate and send a signal back to the control system. The control system can then determine the topology of the lumen at varying depths within the luminary tissue. The imaging system can display this topology for viewing by the medical professional or other user. This technique maps the interior of blood vessels and is valuable in efforts to help treat atherosclerosis by locating blockages caused by occlusive plaque. Once located, these blockages can be removed by a suitable medical procedure such as surgery or angioplasty.

There are, however, medical conditions which cannot be diagnosed by catheter-based imaging or other medical techniques such as angiography or fluoroscopy. One of these conditions is atherosclerosis caused by the formation of vulnerable plaque. Vulnerable plaque sits like an abscess within the vessel wall, covered by a thin membrane or fibrous cap. Although it is asymptomatic and does not constrict arteries, vulnerable plaque is highly susceptible to inflammation. Inflammation, erosion and ulceration can cause vulnerable plaque tissue to rupture. Rupturing of the vessel wall results in an estimated 60-70% of fatal myocardial infarctions. The presence of a large lipid core with associated inflamed tissue is a powerful predicator of potential plaque-induced rupturing.

Thus, there is a need for an improved system and method of imaging lumens and vessel walls.

### SUMMARY

An improved medical device such as catheter preferably includes a transducer configured to receive a thermally generated acoustic wave when the transducer is located within a lumen of a living being and output a first signal corresponding to the intensity of the received thermally generated acoustic wave.

Described next is an example embodiment of an improved catheter. The improved catheter includes an elongated, tubular member having a center axis, a distal end and a proximal end, wherein the transducer is configured to move within the elongated member. The catheter also includes a drive shaft coupled with the transducer and extending proximately from the transducer along the center axis of the elongated member. In one embodiment, the drive shaft can be coupled with the control system, wherein the control system is configured to rotate the transducer radially about the center axis such that the transducer can receive the thermally generated acoustic wave from at least one radial location within the lumen. In another embodiment, the control system can be configured to move the transducer axially along the center axis such that the transducer can receive the thermally generated acoustic wave at at least one axial location of the lumen.

In the example embodiment, the transducer is coupled with an imaging system configured to calculate a temperature of the lumen based on the first output signal. The transducer is coupled with an amplifier configured to amplify the first output signal. The amplifier in turn is coupled with an integrator configured to integrate the amplified signal and output the signal to a processor configured to calculate the temperature of the lumen. In certain embodiments, the temperature of the lumen can be mapped onto an image of the topology of the lumen. The image can be multi-dimensional, color-coded and displayed in near real-time.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1 is a schematic diagram depicting an example embodiment of a thermal acoustic system.

FIG. 2 is a perspective view of an example embodiment of a catheter within the thermal acoustic system.

FIG. 3 is a perspective view of another example embodiment of a catheter within the thermal acoustic system.

FIG. 4 is a cross-section of another example embodiment of a catheter within the thermal acoustic system.

FIG. 5 is a perspective view of another example embodiment of a catheter within the thermal acoustic system.

FIG. 6 is a plot of voltage versus time for an example embodiment of an integrator within the thermal acoustic system.

FIG. 7 is a perspective view of another example embodiment of a catheter within the thermal acoustic system.

FIG. 8A is a plot of voltage versus time for an example embodiment of an integrator within the thermal acoustic system.

FIG. 8B is a plot of voltage versus time for an example embodiment of an integrator within the thermal acoustic system.

FIG. 8C is a plot of voltage versus time for an example embodiment of an integrator within the thermal acoustic system.

FIG. 8D is a plot of voltage versus time for an example embodiment of an integrator within the thermal acoustic system.

FIG. 9 is a perspective view of another example embodiment of a catheter within the thermal acoustic system.

FIG. 10 is block diagram depicting an example embodiment of a method of displaying the temperature of a lumen.

FIG. 11 is block diagram depicting an example embodiment of a method of fabricating a catheter.

### DETAILED DESCRIPTION

The systems and methods described herein provide for the temperature measurement of a lumen of a living body based upon the amount of thermal noise generated by the lumen. In a preferred embodiment, a medical device, such as catheter, is inserted into a lumen of a living body and an acoustic transducer contained within the catheter measures the temperature at positions along the walls of the lumen. For the sake of convenience, reference is made to the example embodiment of a catheter; however, such catheter embodiments can be adapted to be non-catheter embodiments. The catheter is connected to an imaging system where the temperature measurement can be displayed and used in medical diagnosis and treatment. The temperature measurements can be mapped onto a three-dimensional topological, morphological or positional image of the lumen, and color-coded to indicate temperature gradients in the lumen. Large temperature gradients can be indicative of inflamed tissue.

As heat is introduced into a system, the molecules within the system become excited and oscillate in a random chaotic motion. This motion creates weak acoustic waves generally referred to as thermal noise. These waves are emitted over a wide range of frequencies and the intensity of these waves follows a second order relationship to the temperature of the system. Therefore, relatively small changes in temperature can correspond to large changes in wave intensity. The intensity of these waves can be measured and used to calculate the temperature of the system. An acoustic transducer can be configured to measure the intensity of these waves and used to calculate the temperature of the system. Because inflammatory conditions generally create higher temperatures within the affected tissue, the measurement of the temperature variations in tissue can be probative of potential inflammatory conditions such as those caused by vulnerable plaque, infections, malignancies and other diseased or injured tissue.

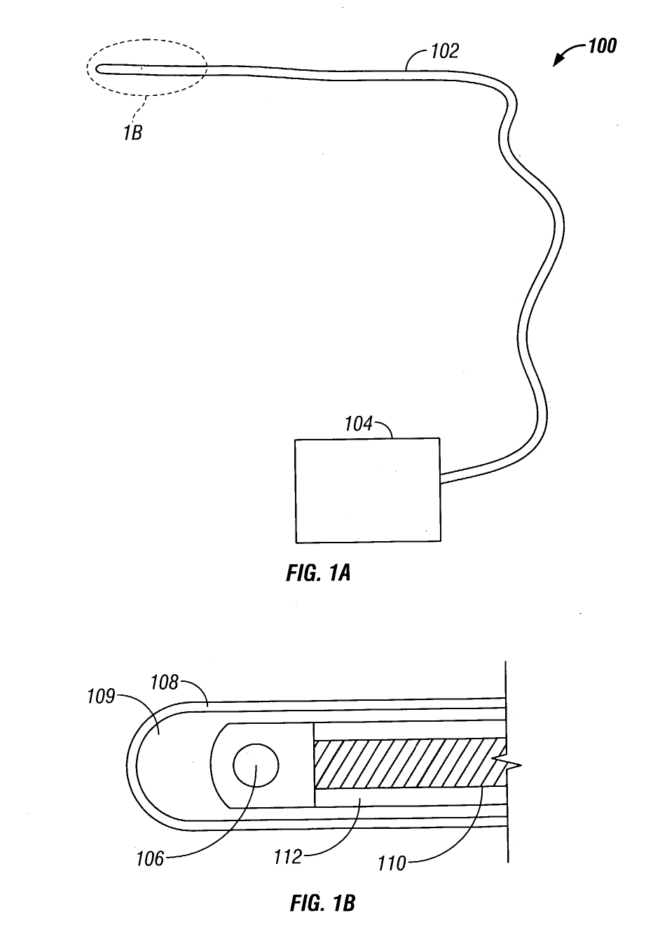

FIG. 1 depicts an example embodiment of a thermal acoustic system 100. Thermal acoustic system 100 is configured to acoustically measure the temperatures within a lumen of a living being and includes catheter 102. Catheter 102 includes transducer 106, elongated member 108, drive shaft 110 and signal line 112. Elongated member 108 is preferably composed of a flexible material and is shaped in a tubular manner, hollowed in the center to allow transducer 106 to move radially and axially within. Transducer 106 may be any kind of known transducer and may be, for example, a forward-facing or side-facing transducer. Transducer 106 is preferably positioned within elongated member 108 at or near the distal end of catheter 102. Connected to transducer 102 is drive shaft 110 and signal line 112, which extend towards the proximal end of catheter 102 where they are coupled with imaging system 104. Of course, as with all embodiments described in this specification, a sonolucent window (not illustrated) and other features known to those of skill in the art of medical imaging and/or catheter design, including for example inflatable balloons and heat-applying devices, may be included. Although the applications and implementations of catheter 102 may vary, in a typical embodiment elongated member 108 is filled with catheter fluid 109, such as saline, and inserted into a lumen of a body, such as a blood vessel or coronary artery.

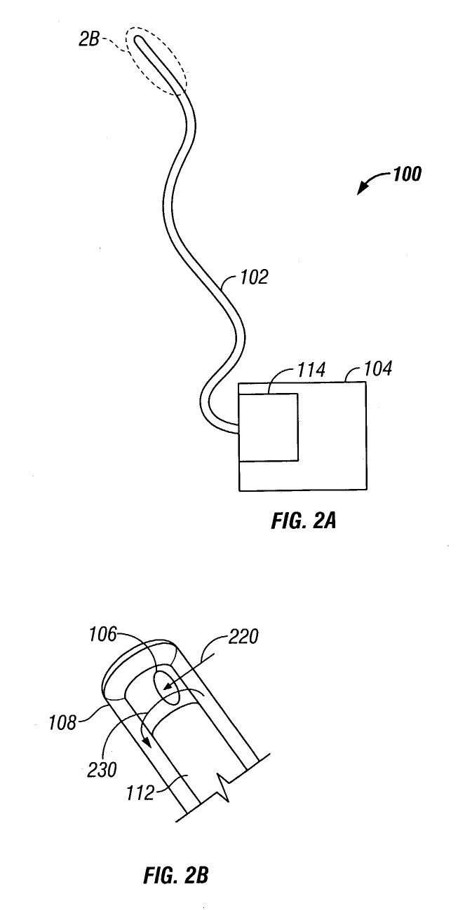

FIG. 2 is a perspective view of one embodiment of catheter 102 within thermal acoustic system 100. In this embodiment, transducer 106 is configured to receive or sense thermal noise uni-directionally, or from one direction, indicated by directional arrow 220. In other embodiments, transducer 106 can be adapted to receive thermal noise in a multi-directional or omni-directional configuration. Imaging system 104 includes control system 114, which operates to rotate drive shaft 110 and transducer 106 radially around center axis 116 of elongated member 108. This radial motion is depicted by directional arrow 230. Control system 114 tracks the radial position of transducer 106, including the orientation of receiving direction 220, which allows each temperature measurement to be correlated with the radial position within the lumen. The rotation of drive shaft 110 thus allows transducer 106 to receive thermal noise at different radial locations within the lumen. This is generally referred to as radially scanning the lumen.

In addition to scanning radially, transducer 106 can also be configured to scan axially along center axis 116 of elongated member 108 as depicted in FIG. 3. To do this, control system 114 operates to pull back (or push forward) drive shaft 110 and with it, transducer 106 in direction 320. This is typically done either at a fixed rate or in step-by-step fashion. Control system 114 again tracks the position of transducer 106 and correlates each measurement to its axial position. If transducer 106 is configured to scan radially in addition to axially, when the pull back occurs in a step-by-step fashion, transducer 106 measures the temperature of the lumen radially at one axial location, and is then pulled back (or pushed forward) to measure radially at a second axial location. When the pull back occurs at a fixed rate, temperature measurements are made while transducer 106 moves radially and axially, resulting in measurements taken along a spiral-like path. Elongated member 108 preferably remains in place while control system 114 moves transducer 106, drive shaft 110 and signal line 112 during radial and axial scanning.

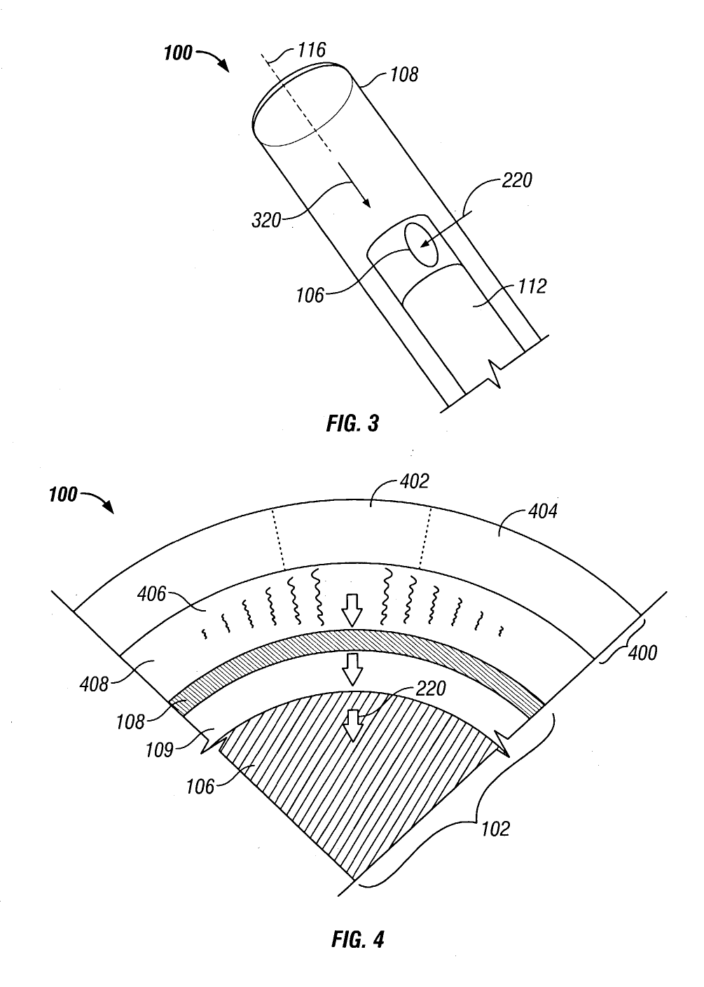

A cross-sectional view of another embodiment of acoustic transducer system 100 is depicted in FIG. 4, showing catheter 102 in proximity with lumen 400. Inflamed region 402 is an area within lumen 400 having a higher temperature than the adjacent non-inflamed region 404. Accordingly, due to the higher temperature, thermal noise 406 generated by inflamed region 402 has a higher intensity than thermal noise 406 generated by non-inflamed region 404. Thermal noise 406, oscillating within the bandwidth of transducer 106 and propagating in direction 220, causes transducer 106 to vibrate. As a result of this vibration, transducer 106 generates output signal 410 (not illustrated), the intensity of which corresponds to the intensity, or strength of, the received thermal noise 406. Output signal 410 is then propagated along signal line 112 (not shown) to imaging system 104 where it is used to calculate the temperature measurement.

In this embodiment, catheter fluid 109 and body fluid 408, which is typically blood, do not substantially interfere with thermal noise 406 because each is a medium that facilitates the propagation of acoustic waves. In another embodiment, catheter 102 is large enough so that it directly contacts lumen 400 and displaces body fluid 408. This eliminates the need for thermal noise 406 to propagate through body fluid 408, and additionally, body fluid 408 can be routed through the interior of elongated member 108 in order to maintain a sufficient level of blood flow. Elongated member 108 is preferably composed of a material chosen so that it does not substantially interfere with the propagation of the acoustic waves. Substantial interference is characterized by reflecting or blocking the passage of the acoustic waves to such a degree that a temperature measurement based on thermal noise 406 reception is not possible.

The precision of the measurements taken by thermal acoustic system 100 is referred to as the temperature resolution (ΔT). The temperature resolution of thermal acoustic system 100 is preferably sufficient to measure the temperature variations between inflamed region 402 and non-inflamed region 404. The degree of temperature resolution required by system 100 may vary depending on the requirements of each individual application; however, a higher resolution is generally favorable. The temperature resolution of acoustic transducer system 100 is a function of time, and more specifically, the temperature resolution of thermal acoustic system 100 can be given by:

$$Δ T = \frac{{T}_{0} \cdot {K}_{N}}{η} \cdot \sqrt{\frac{1}{Δ F \cdot τ} + {( \frac{Δ G}{G} )}^{2}} \qquad ( 1 )$$

wherein:

T₀ is the temperature of the body in Kelvin (K);

K_{N} is the noise coefficient in dB;

η is the efficiency coefficient of transducer 106;

τ is the duration of thermal noise reception in seconds;

ΔF is the bandwidth of transducer 106 in Hertz;

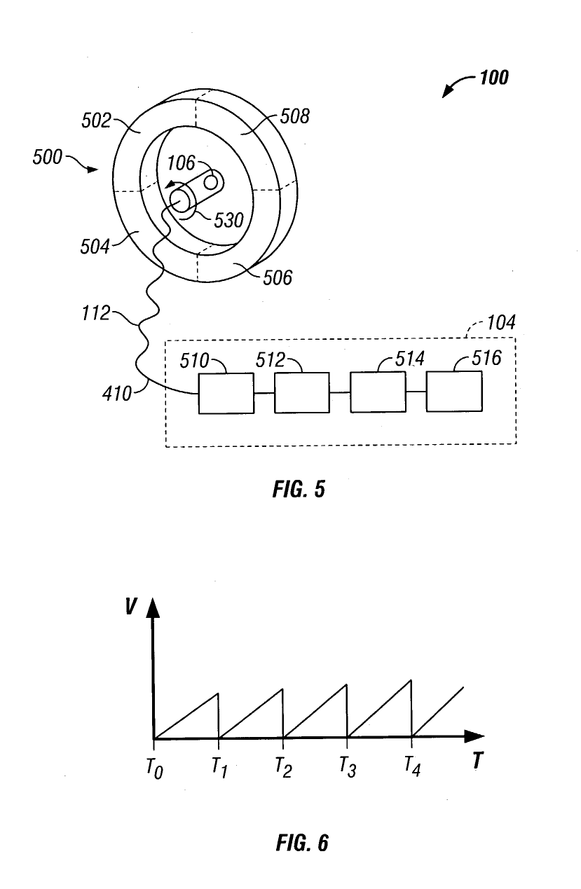

G is the amplification of amplifier 510 (see FIG. 5) in dB; and

ΔG is the fluctuation in amplification of amplifier 510 in dB.

In one embodiment illustrated in FIG. 5, amplifier 510 has little or no fluctuation ΔG, in which case the temperature resolution in (1) could then be given as:

$$Δ T = \frac{{T}_{0} \cdot Kn}{η} \cdot \sqrt{\frac{1}{Δ F \cdot τ}} \qquad ( 2 )$$

Therefore, to achieve a high degree of temperature resolution, i.e. a smaller ΔT, higher values of the efficiency coefficient (η), bandwidth (ΔF) and receiving duration (τ) are desirable, while a lower value of K_{N} is desirable.

Transducer 106 is preferably sensitive enough to receive thermal noise 406 at low intensities. In one embodiment, transducer 106 is a 3F-40 Mhz±8 Mhz transducer and is used to measure temperatures with a desired temperature resolution of less than 0.5 C°. The desired temperature resolution is chosen based on the needs of the each individual application. The size of transducer 106 is chosen based on the size of lumen 400. In this embodiment, a typical transducer 106 size of 3F (French) is chosen and transducer 106 has a resonant frequency of 40 Mhz and a bandwidth of ±8 Mhz, which is 16 Mhz or 40%. The resonant frequency of transducer 106 can vary due to the wide bandwidth of thermal noise 406 and in other embodiments, resonant frequencies can be chosen in the range of 1-2 Mhz.

Applying formula (2) above to an example case, T₀ is 310° K., which is the typical human body temperature in Kelvin, K_{N} is experimentally determined to be 1 dB and the bandwidth of transducer 106 is 16 Mhz. It should be noted that thermal acoustic system is not limited to use in the human body, it can also be used in veterinary or other non-human applications. In one embodiment, the efficiency of transducer 106 is 20% and a resolution of 0.41 C° can be achieved with a duration of approximately 0.898 seconds. In another embodiment, the efficiency of transducer 106 is 30% and allows a resolution of 0.37 C° to be achieved with a duration of only approximately 0.449 seconds. Therefore, the efficiency of transducer 106 can have a significant effect on the duration of reception required to achieve a desired temperature resolution.

FIG. 5 depicts another embodiment of thermal acoustic system 100 where transducer 106 is configured to radially scan cross-section 500 of lumen 400. In this embodiment, transducer 106 rotates within cross-section 500 in direction 530 and receives thermal noise 406 generated by lumen 400. As discussed above, temperature resolution is dependent upon several factors, including transducer efficiency and duration of reception. The duration of reception is determined by the rate of rotation and the time during which transducer 106 is receiving thermal noise 406. Therefore, the rate of rotation, number of rotations, reception time and the efficiency of transducer 106 can be adjusted to achieve a desired level of thermal resolution. In this example embodiment, acoustic thermal system 100 is configured to make four temperature measurements in one rotation of cross-section 500. Accordingly, cross-section 500 is divided into four temperature measurement zones 502, 504, 506 and 508, each corresponding to a separate temperature measurement. The number of temperature measurements made for one rotation will vary depending on the needs of the application.

In this embodiment, acoustic thermal system 100 also includes amplifier 510, integrator 512, processor 514 and display 516, each of which are located within imaging system 104. Also included, but not shown are elongated member 108, drive shaft 110 and control system 114. As transducer 106 rotates, it receives thermal noise 406 and generates output signal 410 corresponding to the intensity of the received thermal noise 406. Output signal 410 is generated by an energy source (not shown) located within transducer 106. Output signal 410 is propagated along signal line 112 to amplifier 510. Amplifier 510 amplifies the received output signal 410 to a higher level to facilitate integration and processing within imaging system 104. In one embodiment, amplifier 510 is configured to amplify output signal 410 while attenuating any unwanted noise. Amplifier 510 can also be configured with a rectifier if desired. In an embodiment where transducer 106 outputs output signal 410 at a sufficient level for processing by imaging system 104, amplifier 510 can be eliminated.

Integrator 512 can be configured to integrate or sum amplified output signal 410 during a temperature measurement and output the integrated output signal 410 once the measurement is complete. In one embodiment, integrator 512 is configured to integrate output signal 410 while ignoring any unwanted noise. FIG. 6 depicts a plot of the voltage integrated on integrator 512 versus time. As transducer 106 rotates, integrator 512 integrates the amplified output signal 410 until the duration of reception for the measurement ends, at which point integrator 512 outputs the integrated output signal 410 to processor 514.

In this embodiment, the measurement for zone 502 begins at time T₀ and ends at T₁. Likewise, the measurements for the other three zones begin and end as follows: zone 504 begins at time T₁ and ends at time T₂; zone 506 begins at time T₂ and ends at time T₃; and zone 508 begins at time T₃ and ends at time T₄, at which point one revolution of cross-section 500 is complete. When transducer 106 begins the rotation through cross-section 500 at T₀, there is no voltage present on integrator 512. As transducer 106 rotates through zone 502, integrator 512 integrates output signal 410 and accordingly, the voltage on integrator 512 rises between T₀ and T₁. At T₁, transducer 106 reaches the end of zone 502 and the temperature measurement is complete. Processor 514 reads out the integrated voltage from integrator 512 and accordingly, the voltage in FIG. 6 is reduced to zero.

The behavior of integrator 512 proceeds in a similar manner for the remaining zones 504-508. As transducer 106 rotates, the temperature measurement for zone 504 is taken and the voltage within integrator 512 rises from T₁ to T₂. The measurement for zone 504 is complete at T₂ and the voltage is again reduced to zero as processor 514 reads the voltage out from integrator 512. This process is repeated for zones 506 and 508 until the temperature of the entire cross-section 500 is measured. T₀ facilitate the illustration in FIG. 6, any change in voltage is depicted as occurring at a constant rate. When there are temperature differences between the zones 502, 504, 506 and 508, the integration results will differ (not shown in FIG. 6). One of skill in the art will readily recognize that the changes in voltage will rarely be at constant rate due to many factors such as variations in the amount of thermal noise 406 received by transducer 106 and noise both external and internal to system 100. In addition, the voltage change when integrator 512 is read out will generally not be instantaneous, but will preferably occur at a high enough rate where the read out time is negligible. As will be discussed below, other embodiments incorporating multiple integrators can compensate for non-negligible read out times.

Based upon the integrated output signal 410, processor 514 calculates the temperature measurement for the corresponding zone 502-508. The temperature measurement is calculated based on the intensity of output signal 410. Higher temperatures typically produce more intense acoustic waves, which in turn cause transducer 106 to generate a more intense output signal 410. The intensity of output signal 410 can then be used by processor 514 to calculate the temperature measurement of lumen 400. Processor 514 may be, for example, a microprocessor, microcontroller, central processing unit (CPU), arithmetic logic unit (ALU), math coprocessor, floating point coprocessor, graphics coprocessor, hardware controller, programmable logic device programmed for use as a controller, or other control logic. Processor 514 may be formed out of inorganic materials, organic materials, or a combination, and may include any of the processing units described in this disclosure or known to those of skill in the art of circuit design. Processor 514 can be any number of processors or controllers capable of calculating the temperature at a rate and accuracy necessary to meet the need of the individual application. Processor 514 can include any additional hardware or software needed for the application, such as facilitating calculations, formatting and mapping data for display and interfacing with control system 114. In a preferred embodiment, processor 514 includes an analog-to-digital converter (ADC) to digitize output signal 408 for processing. Processor 514 outputs the temperature measurement to display 516, which displays the temperature measurement in a format compatible for the user to view.

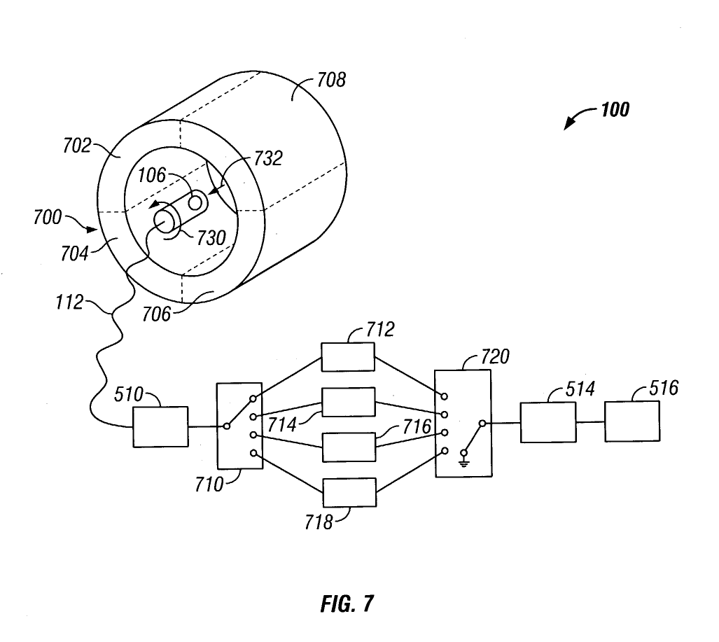

FIG. 7 depicts another embodiment of acoustic thermal system 100 where transducer 106 is configured to scan both radially and axially. Transducer 106 is configured to rotate around cross-section 700 in direction 730 and move axially within cross-section 700 in direction 732. The radial and axial movement can be in a step-by-step fashion or at a fixed rate or in any combination which suits the needs of the application. In this embodiment, the radial and axial movement each occur at a fixed rate. Acoustic thermal system 100 is configured to take four temperature measurements of cross-section 700, depicted as zones 702, 704, 706 and 708.

In this embodiment, transducer 106 radially scans each zone 702-708 multiple times to achieve a desired temperature resolution. Therefore, acoustic thermal system 100 includes four integrators 712, 714, 716 and 718 each corresponding to each of the four zones 702, 704, 706 and 708, respectively. Switch unit 710 connects each integrator 712-718 to amplifier 510 during the temperature measurement of each zone 702-708 being scanned. Switch unit 710 can be any type of switch suitable to meets the needs of the application, such as an electrical, mechanical, or combination electromechanical switch such as a micro-electro-mechanical system (MEMS) switch and may include software. For instance, when transducer 106 scans zone 702, integrator 712 is connected to amplifier 510 by switch unit 710. Once transducer 106 reaches zone 704, switch unit 710 opens the connection with integrator 712 and instead connects amplifier 510 to integrator 714, which remains connected until transducer 106 completes the scan of zone 704. Likewise, when transducer 106 scans zones 706, integrator 716 is the only integrator connected with amplifier 510.

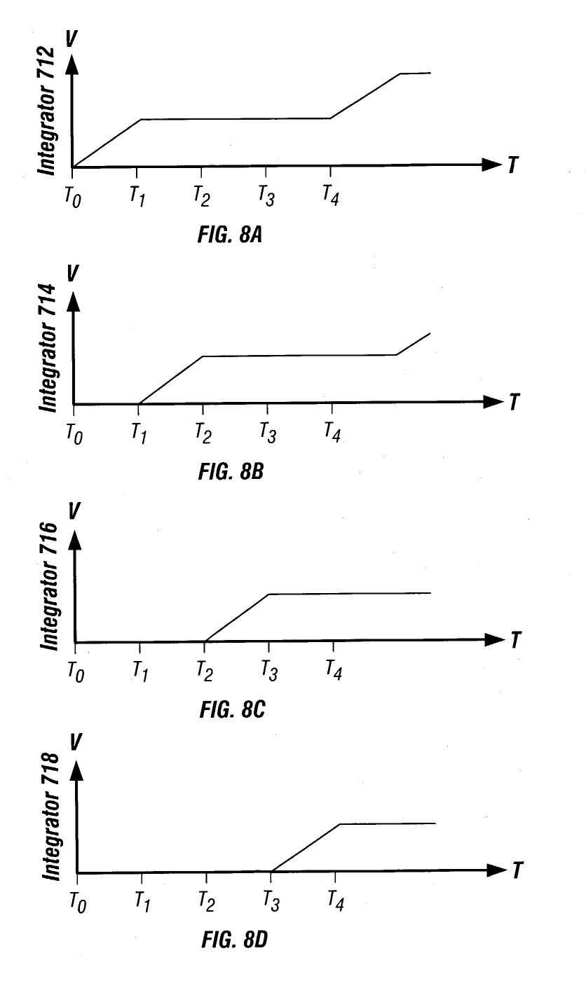

FIGS. 8A-8D depict plots of voltage versus time for each integrator 712-718, respectively, for the embodiment depicted in FIG. 7. At time T₀, transducer 106 begins scanning zone 702 and the voltage of integrator 712 increases. At T₁, transducer 106 completes the scan of zone 702 and begins scanning zone 704. Switch unit 720 disconnects integrator 712 and connects integrator 714 to amplifier 510. Accordingly, the voltage of integrator 712 remains constant and the voltage of integrator 714 rises corresponding with the scan of zone 704. At T₂, the scan of zone 704 is complete and the scan of zone 706 begins. The voltage of integrator 716 rises until T₃, at which point the scan of zone 708 begins and the voltage of integrator 718 rises. At T₄, the scan of zone 708 is complete and transducer 106 begins a second scan of zone 702, and again the voltage of integrator 712 rises. This process repeats until each zone 702-708 is scanned the desired number of times. As discussed earlier, one of skill in the art will readily recognize that the rate of change of the voltages depicted in FIGS. 8A-8D will not necessarily be constant due to the amount of thermal noise 406 received as well as noise internal and external to system 100.

Once each zone 702-708 is scanned the desired number of times, the voltage of each integrator 712-718 is read out by processor 514 through switch unit 720. For instance, when the temperature measurement of zone 702 is complete, switch unit 720 connects integrator 712 to processor 514 so that the voltage of integrator 712 can be read out by processor 514 to calculate the temperature of zone 702. In this embodiment, switch unit 720 preferably only connects one integrator 712-718 at a time to processor 514, and leaves all integrators 712-718 unconnected while the scanning of zones 702-708 is still on-going. Switch unit 720 can be grounded or left open when in an unconnected state. One of skill in the art will readily recognize that other embodiments can be implemented incorporating multiple amplifiers 510 and processors 514 to measure temperature in a radial, axial or combinational manner. In addition, due to the parallel configuration of integrators 712-718, the read out times in this embodiment can be greater than an embodiments implementing just one integrator.

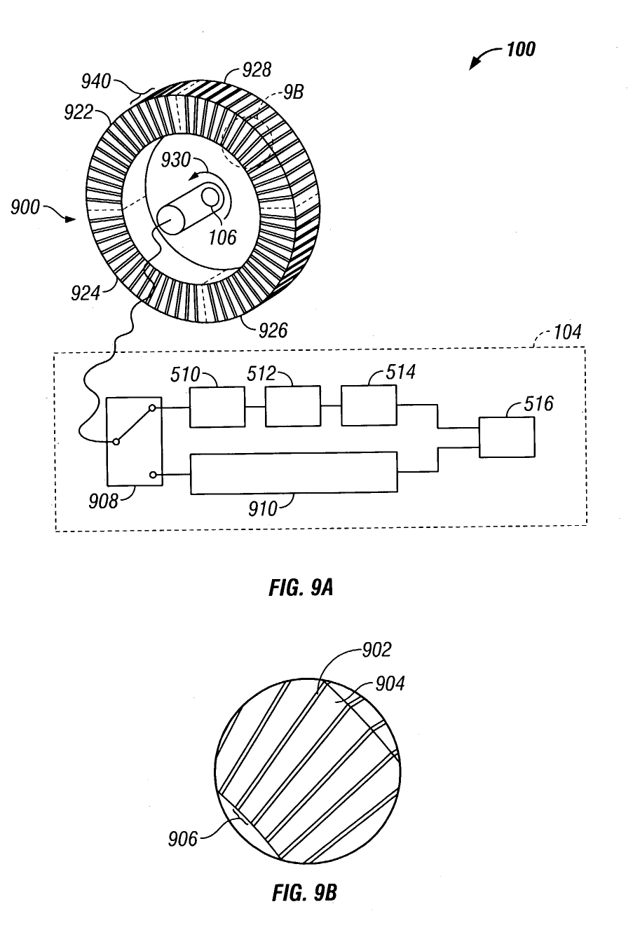

FIG. 9 depicts another embodiment of acoustic thermal system 100 configured to measure the temperature of lumen 400 as well as acoustically scan the topology of lumen 400. In this embodiment, transducer 106 is configured to receive thermal noise 406 and acoustically measure the topology of cross-section 900 during a rotation around cross-section 900 in direction 930. T₀ acoustically measure the topology, acoustic thermal system 100 uses a pulse-echo technique. In one embodiment of the pulse-echo technique, pulse-echo system 910 sends an excitation signal to transducer 106. The excitation signal causes transducer 106 to send an acoustic pulse outwards to the wall of lumen 400. When the acoustic pulse strikes lumen 400, it is echoed back to transducer 106, which then generates an output signal upon receiving the echoed pulse. Pulse-echo system 910 is configured to measure the distance to lumen 400 based upon the time between the transmission and receipt of the acoustic pulse. Pulse-echo system 910 is configured to measure the topology at varying surface depths within lumen 400 by adjusting the frequency of the acoustic pulse transmitted by transducer 106. Typically lower frequencies penetrate further than higher frequencies.

In this embodiment, cross-section 900 is divided into 256 separate zones 906. Each zone 906 includes two zones 902 and 904. Transducer 106 performs a pulse-echo measurement of the topology of lumen 400 while in zone 902 and receives acoustic thermal noise 406 while in zone 904. In this embodiment, each-pulse-echo measurement in a zone 902 can be used as a separate topological measurement, which corresponds to 256 topological measurements for every revolution of cross-section 900. However, to achieve a duration of reception long enough to give the desired temperature resolution, multiple zones 904 are needed for each temperature measurement. In this embodiment, there are four temperature measurement zones 922, 924, 926 and 928 for cross-section 900, similar to the embodiments discussed above. Each temperature measurement zone 922-928 preferably includes 64 zones 904.

The following example further illustrates this embodiment. In this example, transducer 106 rotates at a rate of 26 rotations per second. Cross-section 900 is divided into 256 zones 906. At this rate, the time transducer 106 is in each zone 902 is approximately 15 microseconds, while the time transducer 106 is in each zone 904 is approximately 135 microseconds. In this embodiment, the efficiency of transducer 106 is 30% and the desired temperature resolution is in the range of 0.3-0.4 C°. These values are intended as examples only and do not limit thermal acoustic system 100 in any way. As described in the example above and according to Equation 2, a resolution of 0.37 C° can be achieved with a duration of approximately 0.449 seconds. Transducer 106 is in each zone 922-928 for a total of approximately 8.64 milliseconds. Therefore, 52 revolutions (2 seconds) are needed to achieve a temperature resolution of 0.37 C°.

If transducer 106 is also configured to scan axially, and is pulled back (or pushed forward) at a rate of 0.5 mm/sec, each zone 922-928 has an axial length 940 of 1 mm. Axial length 940 is the distance along the center axis if lumen 400 that transducer 106 travels to obtain one temperature measurement at a given resolution. Preferably, axial length 940 is small so as to allow more axial temperature measurements along lumen 400. However, any decrease in axial length will usually require a reduction in the number of temperature measurement zones in the cross-section if all other factors remain constant. For instance, in another embodiment there are only two temperature measurement zones in cross-section 900. For two temperature measurement zones, only 26 revolutions are needed to achieve the same temperature resolution, and therefore each zone would have an axial length of 0.5 mm.

In this embodiment, acoustic thermal system 100 includes switch unit 908 and pulse-echo imaging system 910 in addition to amplifier 510, integrator 512, processor 514 and display 516. Switch unit 908 connects pulse-echo system 910 when transducer 106 is in zone 902, and conversely, switch unit 908 connects amplifier 510 when transducer 106 is in zone 904. As previously described, switch unit 908 can be any type of switch suitable to meets the needs of the application, such as an electrical, mechanical, or combination electromechanical switch such as a micro-electro-mechanical system (MEMS) switch and may include software.

In this embodiment, thermal acoustic system 100 is configured so that integrator 512 is read out at the end of each zone 904, and processor 514 is configured to sum the integrator 512 values and calculate the temperature measurement for each zone 922-928. However, any number of integrators 512 can be used to meet the needs of the application and indeed, multiple integrators 512 can be used for each of the different temperature measurement zones 922-928. In yet another embodiment, a separate integrator is used for each zone 922-928 if multiple revolutions are needed to achieve the desired resolution. Each integrator could then be read out after completion of the desired number of revolutions.

In this embodiment, the topological measurements and temperature measurements are displayed on display 516. The topological measurements for cross-section 900 can be displayed in either textual or graphical format along with the temperature measurements for the corresponding topological area. For example, in one embodiment the topological and temperature measurements are presented in a raw format, such as a spreadsheet. In another embodiment, an enhanced intraluminal image is generated having a temperature map displayed in conjunction with the image of the lumen, together displaying the morphological and thermometric properties of the lumen.

In this embodiment, multi-dimensional topological image of cross-section 900 is generated and the temperature measurements from each zone 922-928 are mapped onto their corresponding locations in the image. The displayed temperature measurements can be in a multi-dimensional format including two or three-dimensional (2D, 3D) formats and can be generated in or near real-time. The displayed temperature measurements can be color-coded and overlay the topological measurements. The color-coding can correspond with temperature gradients in lumen 400 where large gradients indicate the possibility of inflamed tissue. The topological image can present the information measured by system 100 in any format that facilitates the comprehension of the data by the user in an efficient manner.

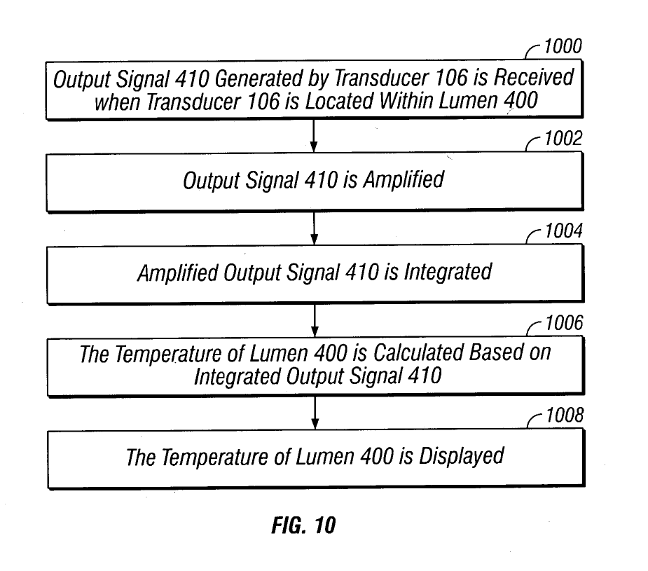

FIG. 10 depicts one embodiment of a method of displaying the temperature of lumen 400. At 1000, output signal 410 generated by transducer 106 is received when transducer 106 is located within lumen 400. Output signal 410 corresponds to the intensity of a thermally generated acoustic wave received by transducer 106. Next at 1002, output signal 410 is amplified, and at 1004 amplified output signal 410 is integrated. Then at 1006, the temperature of lumen 400 is calculated based on integrated output signal 410. Finally, at 1008, the temperature of lumen 400 is displayed.

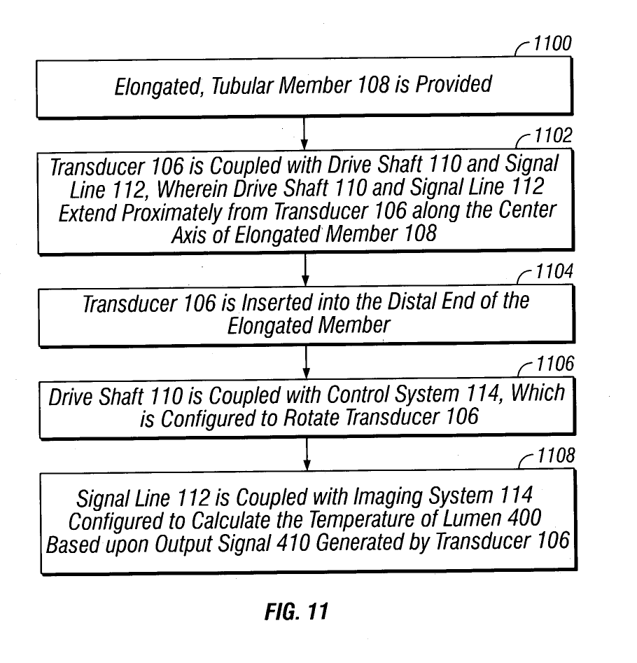

FIG. 11 depicts one embodiment of a method of fabricating catheter 102. At 1100, elongated, tubular member 108 is provided. The elongated member 108 has a distal end and a proximal end. Then at 1102, transducer 106 is coupled with drive shaft 110 and signal line 112, wherein drive shaft 110 and signal line 112 extend proximately from transducer 106 along the center axis of elongated member 108. Next at 1104, transducer 106 is inserted into the distal end of the elongated member. Transducer 106 is configured to receive a thermally generated acoustic wave when the distal end is located within lumen 400. At 1106, drive shaft 110 is coupled with control system 114, which is configured to rotate and/or axially translate transducer 106. Finally, at 1108, signal line 112 is coupled with imaging system 114 configured to calculate the temperature, or determine the relative temperature differences, of lumen 400 based upon output signal 410 generated by transducer 106.

In the foregoing specification, the invention has been described with reference to specific embodiments thereof. It will, however, be evident that various modifications and changes may be made thereto without departing from the broader spirit and scope of the invention. For example, the reader is to understand that the specific ordering and combination of process actions shown in the process flow diagrams described herein is merely illustrative, unless otherwise stated, and the invention can be performed using different or additional process actions, or a different combination or ordering of process actions. As another example, each feature of one embodiment can be mixed and matched with other features shown in other embodiments. Features and processes known to those of ordinary skill may similarly be incorporated as desired. Additionally and obviously, features may be added or subtracted as desired. Accordingly, the invention is not to be restricted except in light of the attached claims and their equivalents.

What is claimed is:

1. A medical imaging device comprising:
an elongated member adapted to be inserted into a lumen of a living being; and
a transducer located within the elongated body, the transducer adapted to receive a thermally generated acoustic wave from the lumen of the living being and to output a first output signal corresponding to the intensity of the received thermally generated acoustic wave.

2. The medical imaging device of claim 1, wherein the transducer is further configured during a first time period to receive a thermally generated acoustic wave and during a second time period generate an acoustic pulse, receive an echo of the generated acoustic pulse and generate a second output signal.

3. The medical imaging device of claim 1, wherein the elongated member comprises a tubular body having a distal end, a proximal end and a center axis, wherein the transducer is configured to move within the elongated member from the distal end to the proximal end along the center axis.

4. The medical imaging device of claim 3, further comprising a drive shaft within the elongated member, the drive shaft coupled with the transducer and extending proximately from the transducer along the center axis of the elongated member.

5. The medical imaging device of claim 4, wherein the drive shaft is coupled to a control system configured to rotate the transducer radially about the center axis such that the transducer can receive the thermally generated acoustic wave from at least one radial location of the lumen.

6. The medical imaging device of claim 4, wherein the drive shaft is coupled with a control system configured to move the transducer axially along the center axis such that the transducer can receive the thermally generated acoustic wave at at least one axial location of the lumen.

7. The medical imaging device of claim 5, wherein the drive shaft is coupled with a control system configured to move the transducer axially along the center axis such that the transducer can receive the thermally generated acoustic wave at one or more axial locations of the lumen.

8. The medical imaging device of claim 1, wherein the transducer is coupled with an imaging system configured to determine relative temperatures of a plurality of portions of the lumen based on a plurality of first output signals, where each of the first output signals is associated with a different portion of the lumen.

9. The medical imaging device of claim 8, wherein the transducer is coupled with an imaging system configured to calculate a temperature of a portion of the lumen based on the first output signal.

10. The medical imaging device of claim 8, wherein the imaging system comprises an amplifier coupled with the transducer, the amplifier configured to amplify the first output signal.

11. The medical imaging device of claim 10, wherein the imaging system comprises an integrator coupled with the amplifier, the integrator configured to integrate the amplified signal.

12. The medical imaging device of claim 8, wherein the imaging system comprises a processor configured to determine the relative temperatures of portions of the lumen.

13. The medical imaging device of claim 9, wherein the imaging system comprises a processor configured to calculate the temperature of the lumen.

14. The medical imaging device of claim 8, wherein the imaging system comprises a display configured to display the relative temperatures of portions of the lumen.

15. The medical imaging device of claim 9, wherein the imaging system comprises a display configured to display the temperature of the lumen.

16. The medical imaging device of claim 2, wherein the transducer is coupled with an imaging system configured to determine the relative temperature of a portion of the lumen based on the first output signal and further configured to acoustically measure a topology of the lumen.

17. The medical imaging device of claim 16, wherein the imaging system is configured to calculate a temperature of the lumen based on the first output signal and further configured to acoustically measure a topology of the lumen.

18. The medical imaging device of claim 16, wherein the temperature is mapped onto an image of the topological measurement of the lumen.

19. The medical imaging device of claim 18, wherein the image is multi-dimensional.

20. The medical imaging device of claim 18, wherein the image is color coded.

21. The medical imaging device of claim 1, wherein the medical imaging device includes a catheter.

22. The medical imaging device of claim 21, wherein the catheter includes a treatment device adapted to treat a portion of the lumen.

23. The medical imaging device of claim 22, wherein the treatment device includes an inflatable balloon.

24. The medical imaging device of claim 22, wherein the treatment device applies heat to the lumen.

25. A method of determining the relative temperature of a first and second portion of a lumen of a living being, the method comprising:
receiving a first thermally generated acoustic wave from the first portion of the lumen of the living being;
generating a first output signal based on the first thermally generated acoustic wave, the first output signal corresponding to an intensity of the first thermally generated acoustic wave;
receiving a second thermally generated acoustic wave from the second portion of the lumen of the living being;
generating a second output signal based on the second thermally generated acoustic wave, the second output signal corresponding to an intensity of the second thermally generated acoustic wave; and
determining the relative temperatures of the first and second portions of the lumen based on the first and second output signals.

26. The method of claim 25, further comprising rotating a transducer radially within the lumen such that the transducer is able to receive the first thermally generated acoustic wave from the first portion of the lumen.

27. The method of claim 25, further comprising moving the transducer axially within the lumen such that the transducer is able to receive the first thermally generated acoustic wave from the first portion of the lumen.

28. The method of claim 25, further comprising:
amplifying the first output signal;
integrating the amplified first output signal; and
calculating a temperature of the lumen based on the integrated first output signal.

29. The method of claim 26, further comprising displaying the temperature of the first portion of the lumen on a display.

30. The method of claim 29, further comprising displaying the temperature of the first portion of the lumen on the display in a color-coded format.

31. The method of claim 29, further comprising displaying the temperature of the first portion of the lumen on the display in a multi-dimensional format.

32. The method of claim 29, further comprising displaying the temperature of the first portion of the lumen on a display in near real time.

33. The method of claim 25, further comprising:
sending an excitation pulse to a transducer configured to cause the transducer to send an acoustic pulse;
receiving a third output signal generated by the transducer, the third output signal generated by the transducer in response to reception of the acoustic pulse echoed by the lumen; and
determining a topology of the lumen.

34. The method of claim 33, further comprising displaying an image of the topology of the lumen.

35. The method of claim 34, wherein the temperature of the lumen is mapped onto the image of the topology of the lumen.

36. The method of claim 25, further comprising treating a portion of the lumen based on the relative temperatures.

37. A method of fabricating a catheter, comprising:
providing an elongated, tubular member having a distal end and a proximal end; and
inserting a transducer into the distal end of the elongated member, the transducer when located within a lumen of a living being is adapted to receive a thermally generated acoustic wave from a portion of the lumen and output a signal based on the intensity of the thermally generated acoustic wave.

38. The method of claim 37, further comprising coupling the transducer with a drive shaft and a signal line prior to inserting the transducer into the elongated member, wherein the drive shaft and the signal line extend proximately from the transducer along a center axis of the elongated member.

39. The method of claim 38, further comprising:
coupling the drive shaft with a control system configured to rotate the transducer; and
coupling the signal line with an imaging system configured to determine the temperature of a portion of the lumen based upon the output signal generated by the transducer.

40. A method of generating an intraluminal image, comprising:
positioning an acoustic transducer within a lumen of a living being;
generating an image of the lumen based upon an energy source from within the transducer; and
generating a temperature map and displaying with the image so that both morphological and thermometric properties of the lumen are displayed.
